(19) United States
(12) Patent Application Publication
(10) Pub. No.: US 2016/0271385 A1
(43) Pub. Date: Sep. 22, 2016

(54) SURGICAL INSTRUMENTS, SYSTEMS, AND METHODS FOR COUPLING OF ELECTRICAL ENERGY TO SURGICAL INSTRUMENTS

(71) Applicant: TransEnterix Surgical, Inc., Morrisville, NC (US)

(72) Inventors: Patrick Sterlina, Apex, NC (US); Galen C. Robertson, Apex, NC (US); Nicholas J. Jardine, Cary, NC (US); Andrew Lane, Morrisville, NC (US); Lucas Tew, Raleigh, NC (US)

(21) Appl. No.: 14/329,887

(22) Filed: Jul. 11, 2014

Related U.S. Application Data
(60) Provisional application No. 62/003,113, filed on May 27, 2014.
(60) Provisional application No. 61/885,231, filed on Oct. 1, 2013.
(60) Provisional application No. 61/844,862, filed on Jul. 11, 2013.

(51) Int. Cl. A61N 1/05

(52) U.S. Cl. CPC A61N 1/05

(57) ABSTRACT

A systems and method for coupling electrical energy to the medical instruments includes a tubular socket having a conductive region and a lumen for receiving a segment of a medical instrument. An energy source is electrically coupled to a conductor in contact with an outer surface of the tubular socket, such that the conductor and the conductive portion of the tubular socket conduct energy to the instrument. The segment of the medical is instrument is rotationally engageable with the tubular socket such that axially rolling of the tubular socket causes axial rolling of a portion of the instrument shaft.

### TECHNICAL FIELD OF THE INVENTION

The present invention relates to conduction of electrical energy to surgical instruments for use in thermal or electrosurgical treatment of tissue.

### BACKGROUND

Many surgical procedures involve the use of instruments having working ends that apply thermal or electrical energy to the tissue in order to cut, dissect, coagulate, ablate, cauterize etc. the tissue. For example, bipolar or monopolar energy may be delivered to the tissue to heat the tissue to achieve the desired effects, or the electrical energy may be used to heat the operative end of the instrument for thermal conduction of heat to the tissue. Such instruments may have operative ends or end effectors in the form of forceps, hooks, blunt dissectors, etc.

Co-pending application Ser. No. 13/759,036, entitled Mechanized Multi-Instrument Surgical System, which is incorporated herein by reference, describes the motor-assisted multi-instrument surgical system 2 shown in FIG. 1. That system 2 includes a finger drive assembly 200 comprising a housing 210 and an insertion cannula 212 extending distally from the housing 210. Steerable instrument delivery tubes or tubular fingers 214 extend distally from the insertion cannula 212. The tubular fingers 214 have lumen for receiving passively flexible surgical instruments 100. As will be described below, motor-driven finger drivers within the finger drive assembly 200 steer the fingers 214 using cables anchored at the distal ends of the fingers. Associated with each tubular finger 214 is a corresponding motor driven roll driver 216—which acts on a distal portion of the instrument shaft to rotate it axially. Both the finger driver and the roller driver are removably mounted to a base which houses motors and electronics for operating the system.

Command interfaces 250 are provided for each of the tubular fingers 214. The command interfaces 250 include instrument boxes 252 that support the instrument handles. The command interfaces 250 are user input devices that generate signals in response to the user's manipulation of the instrument handle (e.g. pan, tilt and roll) and/or other user inputs. In response to signals generated at the command interface 250, the system's motors (housed within the base 218 and having output shafts coupled to corresponding elements in the finger driver and roll driver) are controlled to cause the finger driver and roll driver to drive the fingers and instrument in accordance with the user input.

During use, the fingers 214 and a portion of the insertion tube 212 are positioned through an incision into a body cavity. The distal end of a surgical instrument 100 is manually, removably, inserted through an instrument box 252 of command interface 250, and the corresponding roll driver 216 and into the corresponding tubular finger 214 via the finger drive assembly 200. The instrument is positioned with its distal tip distal to the distal end of the tubular finger 214, in the patient's body cavity, and such that the handle 104 of the instrument is proximal to the command interface 250.

The user manipulates the handle 104 in an instinctive fashion, and in response the system causes corresponding movement of the instrument's distal end. The motors associated with the finger driver are energized in response to signals generated when the user moves the instrument handles side-to-side and up-down, resulting in motorized steering of the finger and thus the instrument's tip in accordance with the user's manipulation of the instrument handle. Combinations of up-down and side-side motions of an instrument handle will steer the instrument's tip within the body cavity up to 360 degrees. Manual rolling of the instrument handle about the instrument's longitudinal axis (and/or manually spinning of a rotation knob or collar proximal to the instrument handle) results in motorized rolling of distal part of the instrument's shaft 102 (identified in FIG. 11) using the roll driver 216.

Additional features of the roll driver 216 disclosed in the prior application will be described with reference to FIGS. 1-7. The roll driver 216 (FIGS. 1 and 2) includes a housing 217. As shown in FIG. 3, a roll drive tube 248 is axially rotatable within the roll driver housing 217 (not shown in FIG. 3). Roll drive tube 248 includes a lumen for receiving a portion of the shaft of instrument 100 (FIG. 1). The exterior of the roll drive tube 248 forms a worm gear, which engages with a roll gear assembly that includes an adjacent worm gear 249. The roll gear assembly includes a member such as driven roll shaft 234 that is exposed at the lower surface of the housing 217 (not shown). The driven roll shaft 234 is axially rotatable relative to the roll driver housing 217.

The roll driver 216 is positionable on the base unit 218 such that the driven roll shaft 234 rotationally engages with a motor-driven drive shaft within the base unit 218. This rotational engagement allows transfer of torque from the motor-driven drive shaft to the shaft 234—thus allowing rotation of the roll drive tube 248 (and thus the instrument shaft) through activation of the roll motor 238.

Openings at the proximal and distal surfaces of the roll driver housing 217 allow passage of an instrument shaft through the lumen of the roll drive tube 248. The roll drive tube 248 has features designed to rotationally engage with corresponding features on the surgical instrument shaft. This engagement allows axial rotation of the roll drive tube 248 to produce axial rotation of the distal portion of the instrument shaft. Preferred features are those that create rotational engagement between the instrument shaft and the roll drive tube 248, but not sliding or longitudinal engagement. In other words, the features are engaged such that axial rotation of the roll drive tube 248 axially rotates the instrument shaft, but allow the instrument to be advanced and retracted through the roll drive tube 248 for “z-axis” movement of the instrument tip. Rotational engagement between the instrument shaft and the roll drive tube 248 should preferably be maintained throughout the useful range of z-axis movement of the instrument tip (e.g. between a first position in which the instrument tip is at the distal end of the finger to a second position in which the instrument tip is distal to the distal end of the finger by a predetermined distance.)

Engagement features for the instrument 100 and roll drive tube 248 include first surface elements on a drive segment 260 of the shaft 102 of the instrument 100 (FIG. 4A) and corresponding second surface elements on the inner surface of the roll drive tube 248 (FIG. 3). Examples of surface elements 256, 258 are shown in FIGS. 5A-7. Referring to FIGS. 5A and 5B, the drive segment 260 of the instrument shaft 102 includes first surface elements 256 in the form of splines or ribs extending radially from the instrument shaft and longitudinally along the shaft. The lumen of the roll drive tube 248 includes second surface elements 258 in the form of longitudinally extending ribs (also visible in FIG. 5B). The surface elements 256, 258 are positioned such that when the roll drive tube 248 is rotated, second surface elements 258 on the interior lumen of the roll shaft contact and cannot rotationally bypass the surface elements on the instrument shaft. The distal ends of the splines 256 may be tapered such that they are narrower (in a circumferential direction) at their distal ends than they are further proximally, to facilitate insertion of the splines/ribs between corresponding ones of the ribs while minimizing play between the splines 256 and adjacent ribs 258 as the roll shaft rotates the instrument shaft. The longitudinal length of the splines 256 is selected to maintain rotational engagement between the instrument shaft and the roll shaft throughout the desired z-axis range of motion. The drawings show three splines spaced 120 degrees apart, although other numbers of splines may be used, including four splines spaced 90 degrees apart.

The drive segment 260 of the instrument shaft may have a larger diameter than proximally- and distally-adjacent sections, as shown in FIG. 4A. To facilitate insertion of the drive segment 260 into the roll drive tube 248, the drive segment 260 includes a chamfered distal edge 262.

As another example, shown in FIGS. 6A-6C, the drive segment 260 has a hexagonal cross-section and the roll drive tube 248 has longitudinal grooves with v-shaped radial cross-sections as shown. Edges 256a of the drive segment 260 formed by corner regions of the hexagonal cross section seat in troughs 258a so as to permit longitudinal sliding of the instrument through the lumen but prevent rotation of the instrument within the lumen.

In another embodiment shown in FIG. 7, drive segment 260 includes longitudinally extending grooves 256b. One or more pins 258b extend radially inwardly from the luminal wall of the roll drive tube 248 and into engagement with one of the grooves 256b.

It should be noted that the instrument 100 is preferably constructed so that the roll drive tube 248 will cause rolling of the drive segment 260 and all portions of the instrument shaft 102 that are distal to it (including the end effector), without causing axial rolling of the instrument handle 104. Thus the handle and shaft are coupled together in a manner that permits the instrument shaft to freely rotate relative to the handle when acted upon by the roll drive tube 248. For example, the instrument 100 might includes a roll joint within, or proximal to, the drive segment.

In some cases, the instrument 100 to be used with such a roll driver is one whose operation requires that electrical energy be coupled from an electrosurgical unit or generator to conductors within the instrument 100, so as to energize electrodes in or on the operative end for electrosurgical treatment or thermal heating of tissue. When conventional instruments are used, cords are attached between the instrument handle and an energy source in order to provide energy to instruments. However, cords extending between the instrument handle and an energy source create additional clutter within the surgeon's working area and can interfere with the surgeon's manipulation of the instrument's handle. The present application therefore discloses systems and methods for coupling electrical energy to the medical instruments without the need for a cord extending to the instrument handle.

### BRIEF DESCRIPTION OF THE DRAWINGS

FIG. 1 is a perspective view of a motor-assisted multi-instrument surgical system.

FIG. 2 is a perspective view of a roll driver of the system of FIG. 1.

FIG. 3 is a perspective view of the roll drive tube and gear assembly of the roll driver.

FIG. 4A is a side elevation view of an instrument that may be used with the system.

FIG. 5A is a perspective view of the drive segment of the instrument of FIG. 5A.

FIG. 5B shows the drive segment of FIG. 5A positioned within the roll drive tube.

FIGS. 6A and 6B are end views of an alternative roll drive tube and drive segment, respective.

FIG. 6C shows rotational engagement of the roll drive tube and drive segment of FIGS. 6A and 6B.

FIG. 7 shows rotational engagement of a second alternative roll drive tube and drive segment.

FIG. 8 shows an instrument partially inserted into an exemplary roll driver. The instrument's handle is not shown.

FIG. 9 is a perspective view showing the roll socket and leaf spring conductors of the embodiment of FIG. 8, and further shows the roll connector of the instrument.

FIG. 10 is a perspective view of the roll connector of the instrument.

FIG. 11 is an end view of the roll socket, the roll connector within the socket, and the leaf spring conductors in contact with the exterior of the roll socket.

FIG. 12 is a perspective view of an alternative embodiment of a roll driver.

FIG. 13 is similar to FIG. 12 but the wheel and connectors are not shown.

FIG. 14 shows the leaf spring conductors and associated insulator shown in FIG. 13. This insulator is shown as transparent to allow the leaf spring conductors to be more easily seen.

FIG. 15 shows the FIG. 12 embodiment with the housing removed.

FIG. 16 is similar to FIG. 15, but also does not show the support.

FIG. 17 is a perspective proximal end view of the components shown in FIG. 16.

FIG. 18 is a bottom perspective view of the roll driver of FIG. 12.

FIG. 19 is a perspective view of the roll drive tube.

FIG. 20 is an exploded view of the roll drive tube of FIG. 19.

FIG. 21 is a perspective view of the insulative tube portion of the roll drive tube shown in FIG. 20, rotated slightly to show both windows.

FIGS. 22 through 27 show an alternative roll drive tube, in which:

FIG. 22 is a perspective view showing an tubular insulator on the roll drive tube halves.

FIG. 23 is a perspective view of the tubular insulator.

FIG. 24 is similar to FIG. 23 but shows the tubular insulator rotated approximately 180 degrees.

FIG. 25 is similar to FIG. 22 but shows the ring conductors on the tubular insulator.

FIG. 26 is similar to FIG. 25, but shows the roll drive tube fully assembled, with the insulating rings positioned adjacent to corresponding ring conductors.

FIG. 27 illustrates contact between the leaf spring conductors and the roll drive tube of FIGS. 22-26.

FIGS. 28-29 are an end view and a side view, respectively, showing an alternative embodiment of a drive segment in which conductive leaf springs are used to electrically couple the drive segment to the roll drive tube.

FIG. 30 is similar to FIG. 28 but shows the drive segment disposed in a roll drive tube.

### DETAILED DESCRIPTION

The present application describes systems and methods for coupling electrical energy to the medical instruments. The medical instruments may be of the type having end effectors that deliver energy to tissue, or that utilize energy for some other purpose. In the embodiments disclosed herein, a roll driver 216, which may be of the type disclosed in the Background section, is used to conduct energy from the energy source (electrosurgical unit/generator) to a conductive surface on the instrument. The instrument is constructed such that the conductive surface is in electrical contact with conductors that extend along or through the instrument shaft, so as to conduct energy to the end effector or other type of element that makes use of energy. The roll driver 216 is thus configured to both connect electrical energy to the instrument and to cause rolling of the instrument shaft.

FIG. 8 schematically shows instrument 100 partially inserted into a first embodiment of a roll driver 216, with the splined drive segment or roll connector 260 of the instrument positioned just proximally of the roll drive tube 248. The proximal part of the instrument, including the handle, is not shown.

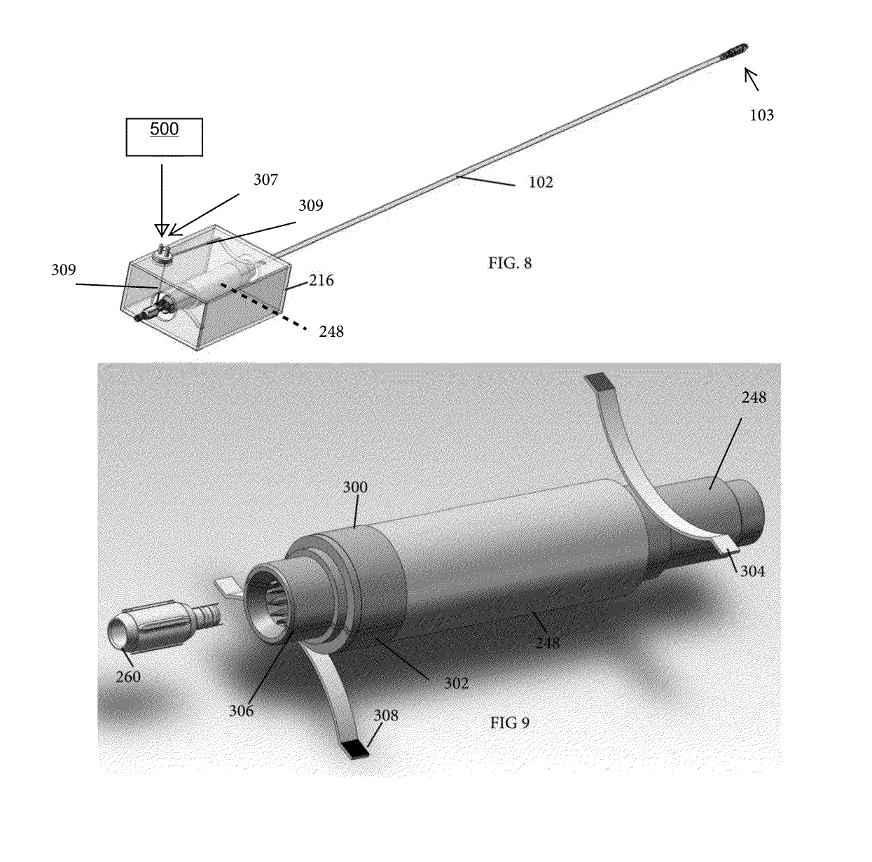

Referring to FIGS. 9 and 11, the roll drive tube 248, or roll socket, is a cylindrical tube formed of two semi-cylindrical conductive halves 300, 302 that are electrically insulated from one another by insulating strips of material 306 running the length of the socket. Socket halves 300, 302 may be made of stainless steel or other suitable electrically conductive material.

As more easily seen in FIG. 9, a pair of spring leaf conductors 304, 308 are provided, each biased in contact with the exterior surface of the roll socket 248 such that as the roll socket 248 rolls axially, these conductors remain in sliding contact with its outer surface. Each conductor 304, 308 is electrically connected to an external surgical energy source, which in this embodiment is a bipolar source 500. Thus, each leaf spring forms a supply/return path (in contact with one half of the roll socket) for the bipolar energy source.

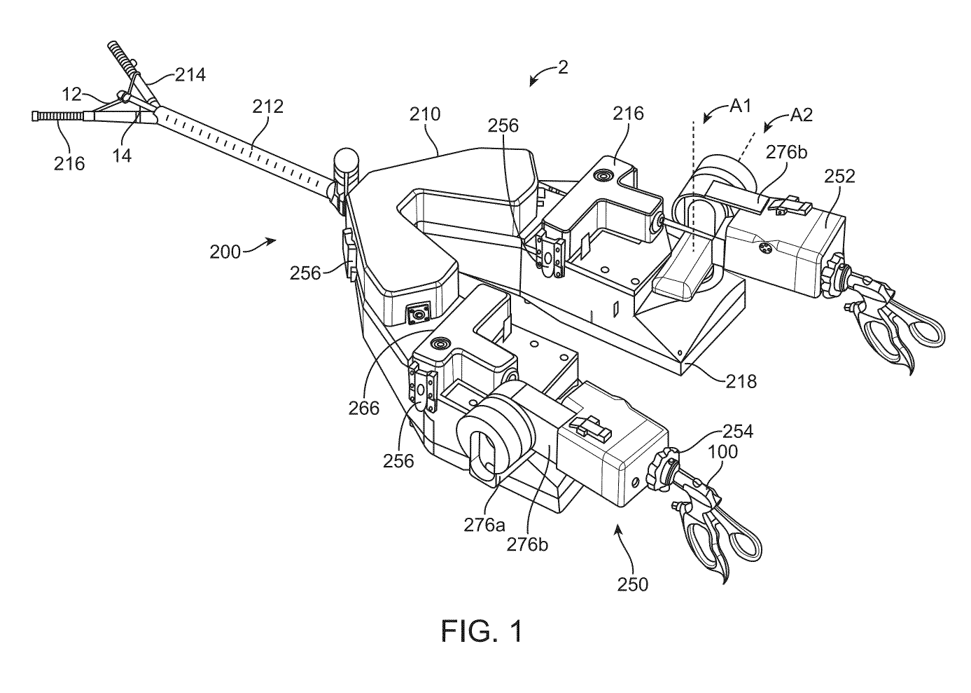

FIG. 8 illustrates a lead connector 307 on the housing of the roll driver 216. The lead connector 307 detachable receives a cord (illustrated schematically) that extends to the external energy source 500. Leads 309 extend between the lead connector 307 and the conductors 304, 308. The external source might instead be coupled elsewhere, such as to the base 218 (FIG. 1). In such an embodiment, conductors on the base would be positioned to conduct energy between conductors in the base and the leaf springs of the roll driver 216. The two conductors 304, 308 are positioned 180 degrees apart in the assembly shown, such that each contacts a different conductive half 300, 302 of the roll socket assembly 248 at any given time.

In the embodiment shown, conductor 308 might serve as the supply side of the energy source and conductor 304 the return. As the roll socket 248 axially rotates to roll the instrument, only one of the socket halves 300, 302 is in contact with either conductor 304,308 at any moment. As the roll socket rotates on an axis parallel to its length, the two halves of the roll socket can freely alternate between serving as the energy supply or return.

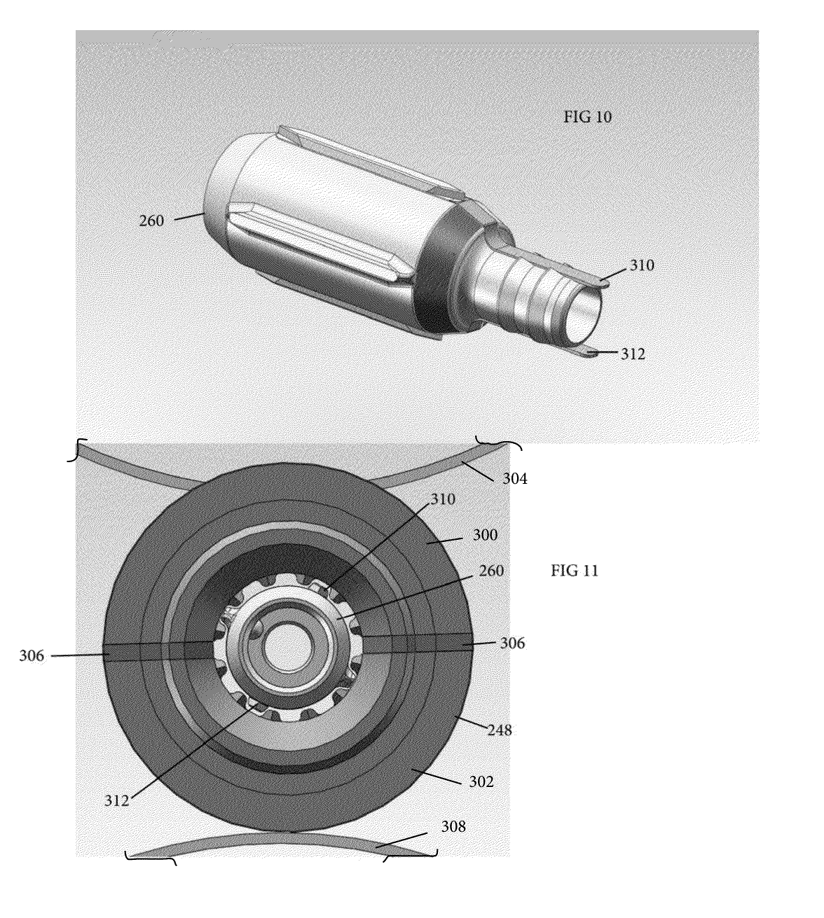

Referring to FIG. 10, the drive segment 260 of the instrument shaft 102, which may also be referred to as the roll connector, has two isolated electrical leads 310, 312 that run the length of the instrument shaft 102 down to the end effector 103. The end effector in this embodiment includes two jaws electrically isolated from each other as seen in typical bipolar surgical instruments—the jaws may serve as, or carry, electrodes. The electrodes may be positioned to conduct electrical energy to tissue, or to resistively heat so as to deliver thermal energy to the tissue.

Leads 310, 312 terminate on separate contact splines/fins on the outer diameter of the roll connector 260. These fins, in turn, contact the drive splines on the inner diameter of the roll socket 248 (with each of the leads 310, 312 in contact with or otherwise electrically coupled to a different one of the socket halves 300, 302) as shown in FIG. 11, thus creating an electrical path through the socket down the leads that run the length of the instrument shaft into the end effector jaws, through the tissue and back up the opposing instrument shaft lead into the opposite contact fin. With this configuration, the end effector may be energized any time the instrument is positioned with the drive segment 260 disposed within the roll socket, including during rotation of the instrument using the roll driver 216 or z-axis movement of the instrument 100.

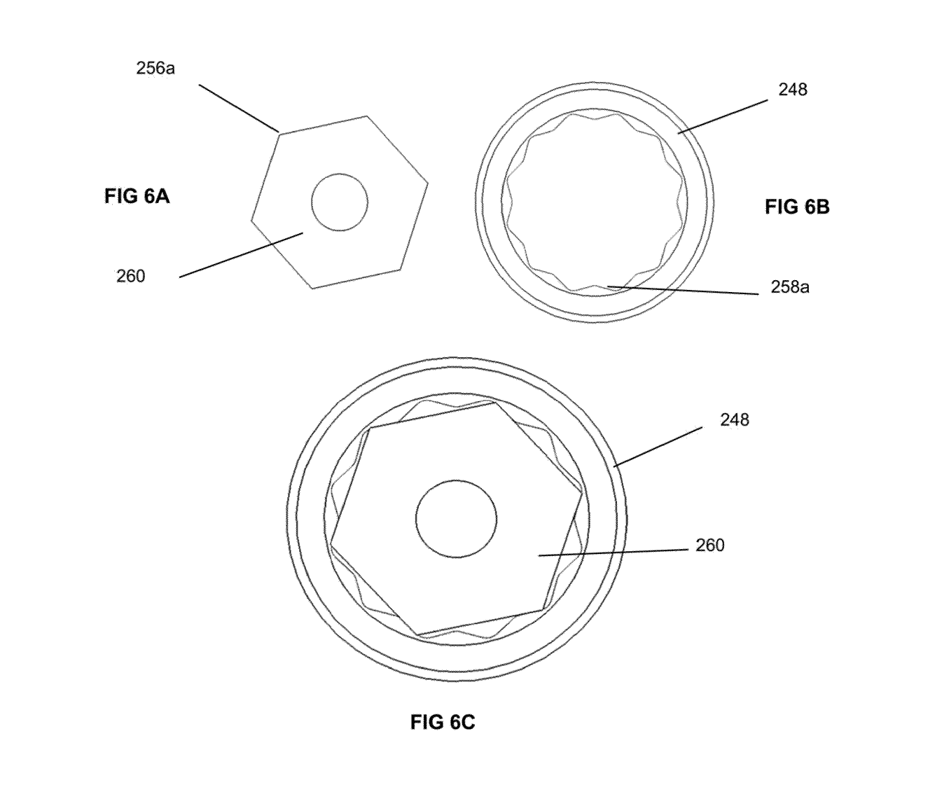

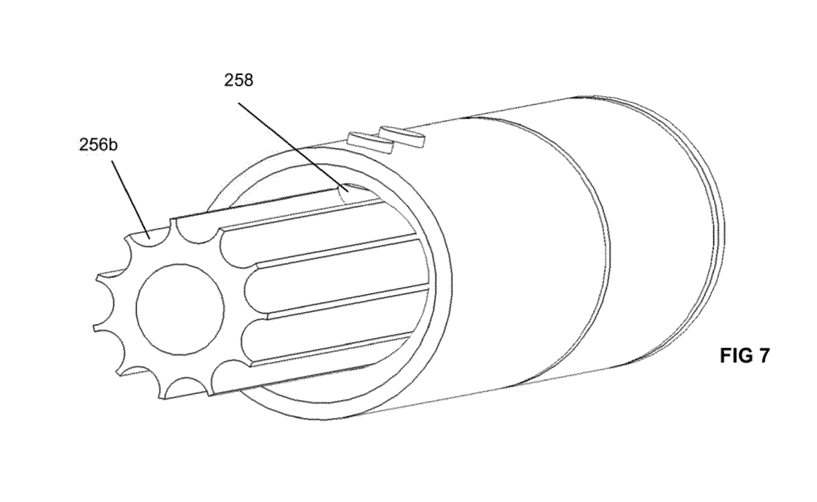

Although the illustrated embodiment makes use of the drive segment 260 and roll socket 248 configurations of FIGS. 5A and 5B, the design may be modified for use with other drive segment/roll socket combinations, including those shown in FIGS. 6A through 7.

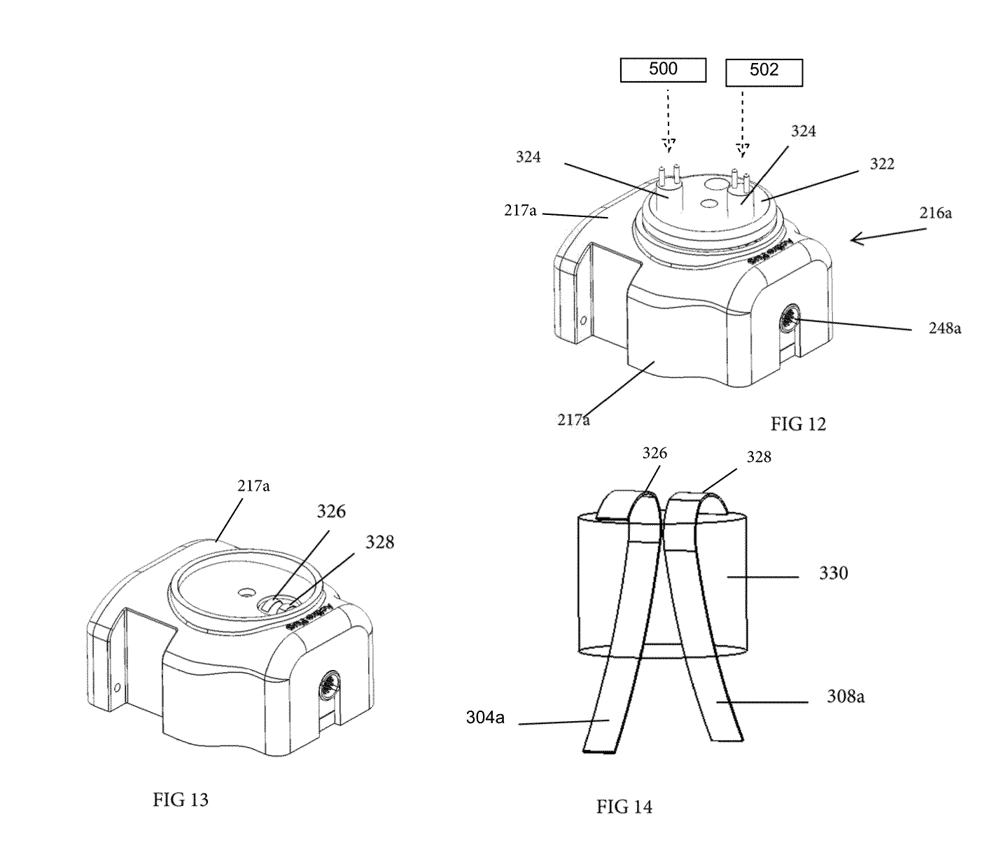

An alternative embodiment of a roll driver 216a that may be used with the instrument 100 is shown in FIGS. 12 through 21. Referring to FIG. 12, roll driver 216a includes a housing 217a and a wheel 322 positioned on the housing 217a. At least two connectors 324 (but optionally more than two) having conductive prongs are disposed on the wheel. Each connector 324 is configured to be electrically coupled to a separate energy source 500, 502 (e.g. a mono-polar energy source, a bi-polar energy source, a generator for devices that treat tissue with thermal energy that may be generated through resistive heating, and/or vessel sealing devices etc.) such as through the use of cables each of which is detachable connected between one of the connectors 324 and an energy source 500, 502.

Beneath the wheel are a pair of exposed contacts 326, 328, as shown in FIG. 13. The wheel 322 is rotatable relative to the housing 217a to selectively position a select one of the connectors 324 in alignment with the contacts 326, 328 such that the lower end (not shown) of each of the corresponding prongs is in electrical contact with one of the contacts 326, 328. The non-selected contacts remain electrically isolated from the roll driver and thus from the instrument 100. Thus, a user may leave multiple energy sources connected to the wheel 322 at one time. In such cases, the user need only to turn the wheel in order to electrically couple the energy source needed for the instrument to be used through the roll driver. When a first instrument requiring a first energy source is to be replaced with a second instrument requiring a second energy source, the wheel is rotated to electrically couple the second energy source to contacts 326, 328 (and to thereby electrically de-couple the first energy source from contacts 326, 328). The first instrument is withdrawn from the roll driver and the second instrument is positioned through the roll driver. Detents (not shown) may be positioned between the wheel and the housing to frictionally retain the wheel in the selected position until it is actively rotated to an alternate position by the user.

Where the energy source does not require multiple energy channels (e.g. a monopolar device), a connector 324 may be used that electrically shorts the contacts 326, 328.

It can be seen in FIG. 14 that the contacts 326, 328 are portions of a pair of leaf spring conductors 304a, 308a. As with the leaf spring conductors of the prior embodiment, each leaf spring conductor 304a, 308a includes a free end that (as is discussed below) is disposed in contact with a portion of the roll drive tube 248a for conduction of electrical energy thereto.

With continued reference to FIG. 14, leaf spring conductors 304a, 308b extend through and are supported by an insulative element 330 (shown as transparent in FIG. 14), such that the contacts 326, 328 are exposed at one end of the insulative element, and such that the opposite ends of the leaf spring conductors 304a, 308b extend from a separate portion of the insulated element (such as its opposite end as shown in FIG. 14). The illustrated insulative element may be a generally cylindrical member as shown, or it may have any shape that maintains the spring conductors 304a, 308b electrically isolated from one another.

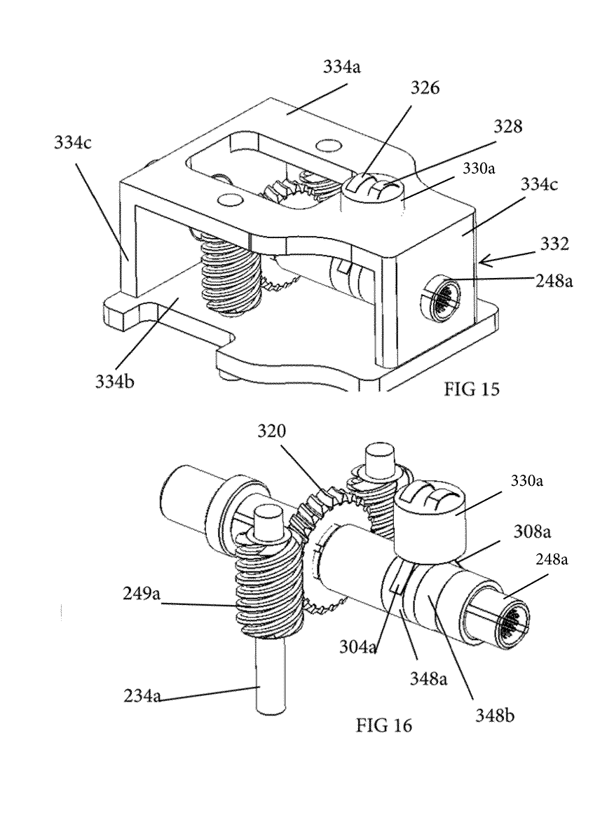

Referring to FIG. 15, the roll driver 216a includes a structural support 332 within the housing 217a (not shown). The structural support 332 includes a top plate 334a, base plate 334b, and end walls 334c. The base plate 334b, together with the ceiling and surrounding walls of the housing 217a, forms an enclosure. The housing 217a may be detachable from the support 332 between uses to allow the interior of the enclosure and its contents to be more thoroughly cleaned and sterilized.

Roll drive tube 248a is at least partially disposed within the enclosure, preferably with its proximal and distal portions supported by end walls 334c of the structural support 332.

Insulative element 330 is disposed through an opening in the top plate 334a of the support 332 with contacts 326, 328 exposed and with the opposite ends of the spring conductors 304a, 308a extending into contact with the roll drive tube 248a.

As with the embodiment described with reference to FIG. 3, the roll drive tube 248a shown in FIGS. 15 and 16 is axially rotatable within the roll driver housing 217a (not shown in FIGS. 15 and 16) and relative to the support 332. Roll drive tube 248a has a lumen for receiving, and rotationally engaging with, a portion of the shaft of instrument 100 (FIG. 1). A gear 320 is positioned on the exterior of the roll drive tube 248a. A roll gear assembly includes a roll shaft 234a having a first end supported by the upper plate 334a, a second end extending through an opening in the base plate 334b of the support 332, and a worm gear 249a disposed between the first and second ends. As described with respect to FIG. 3, the end of the shaft 234a is exposed at the lower surface of the housing 217a (not shown). The shaft 234a and its gear 249a are rotatable about to their longitudinal axis relative the roll driver housing 217a and the support 332.

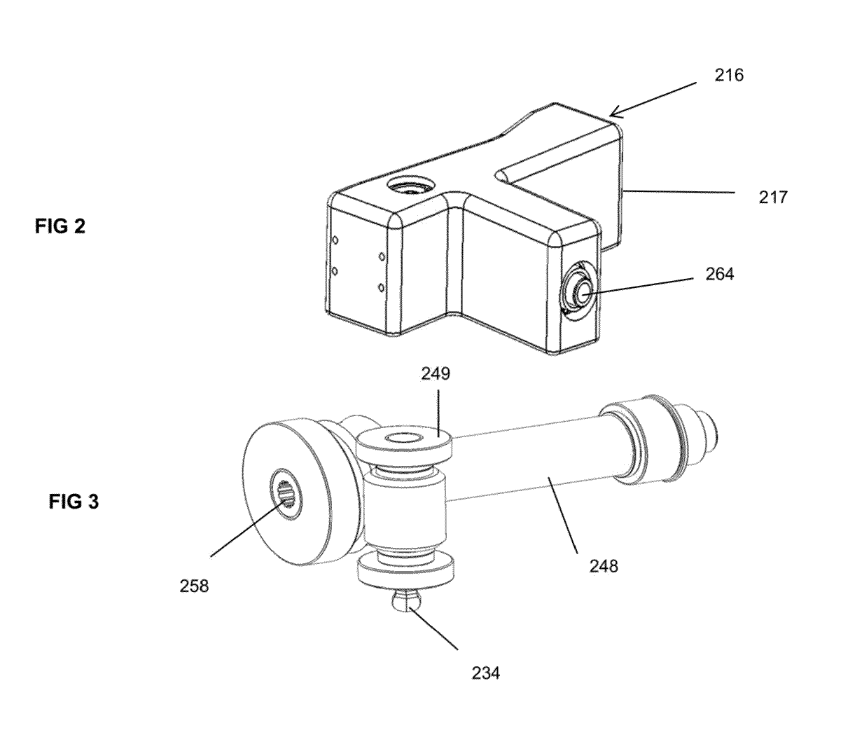

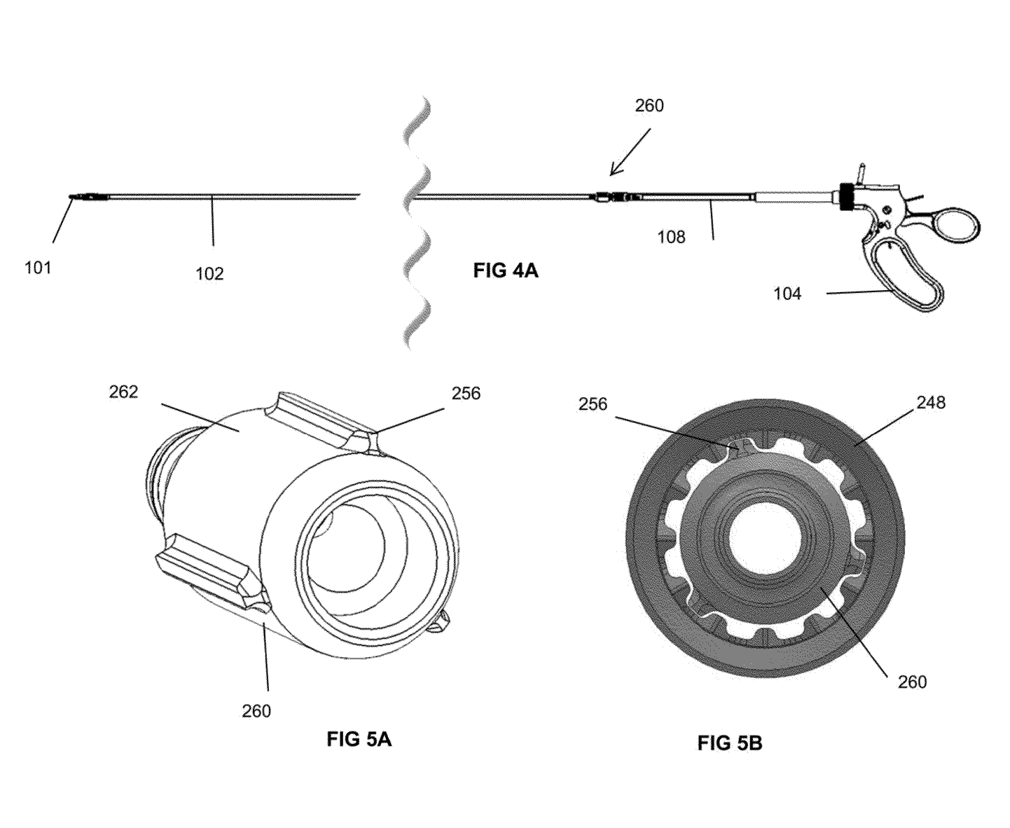

As discussed with reference to FIGS. 1 to 3, the roll driver 216a is positionable on the base unit 218 such that the roll shaft 234a rotationally engages with a motor-driven drive shaft within the base unit 218. This rotational engagement allows transfer of torque from the motor-driven drive shaft in the base unit 218 to the shaft 234a of the roll drive 216a—thus allowing rotation of the roll drive tube 248a (and thus the instrument shaft extending through it) through activation of the roll motor in the base unit. The shaft 234a includes a hex head (as shown in FIG. 3) or alternative structure for rotational engagement with the motor driven drive shaft of the base unit 218.

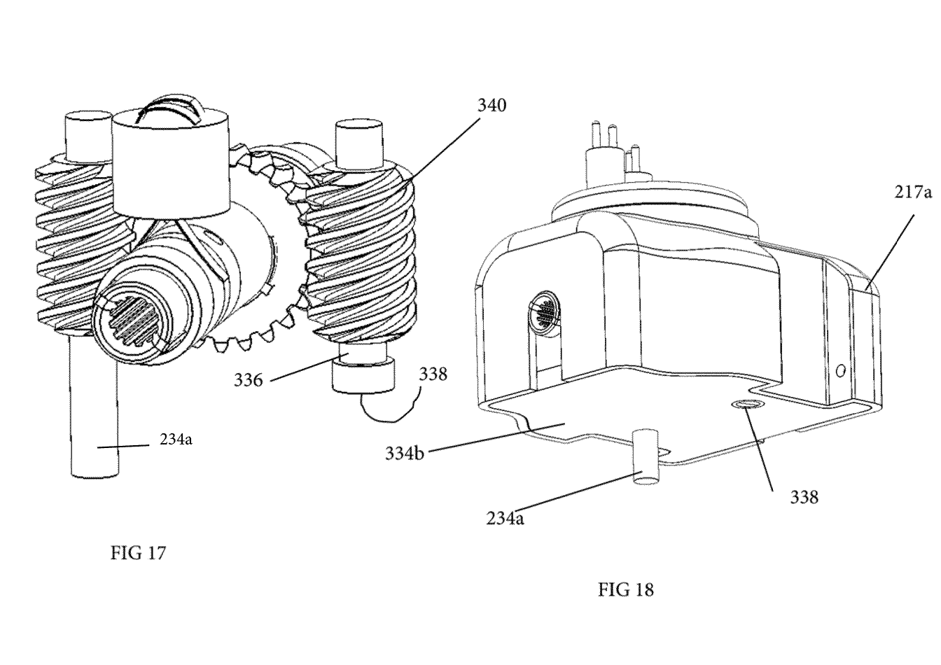

As shown in FIG. 17, a second shaft 336 is provided having a first end supported by the upper plate 334a and a second end disposed in an opening in the base plate 334b of the support 332. A magnet 338 is disposed at the second end and exposed through base plate 334b as shown in FIG. 18. The magnet 338 includes diametrically positioned north and south poles and preferably faces downwardly towards the base 218 when the roll driver 216a is disposed on the base. Encoder chips on the base 218 are positioned to align with the magnet 338 when the roll driver 216a is mounted on the base 218.

A second worm gear 340 disposed between the first and second ends. When the shaft 234a is rotated to cause axial roll of the instrument (through action of worm gear 249a on gear 320), the second worm gear 340 causes the second shaft 336 to rotate, thus rotating the magnet 338. The encoder chip in the base senses the rotational position of the nearby magnet 338. This information allows the system to detect how many degrees the instrument has rolled relative to its initial position. Signals generated by the encoder chips may also be used by the system to detect when each roll driver 216a has been mounted to the base 218.

Referring to FIG. 20, the roll drive tube 248a is a cylindrical tube formed of two semi-cylindrical conductive halves 300a, 302a that are electrically insulated from one another by insulating strips 306 of material running the length of the tube as discussed with respect to the prior embodiment. Conductive halves 300a, 302a may be made of stainless steel or other suitable electrically conductive material.

As discussed above, the leaf spring conductors 304a, 308b are positioned in contact with the roll drive tube 248a so as to conduct electrical energy to the roll drive tube, which further conducts the electrical energy to the instrument extending through it. The roll drive tube 248a is provided with insulating members arranged such that electrical energy from one of the leaf spring conductors passes only to one of conductive halves, and such that electrical energy from the other one of the leaf spring conductors passes only to the other of the conductive halves, despite the fact that the leaf spring members maintain constant contact with the roll drive tube 248a throughout its rotation.

Referring again to FIG. 20, the roll drive tube 248a includes a mount section 342 on which the gear 320 (FIG. 16) is mounted. An insulating tube 344 is positioned over a portion of the roll drive halves 300a, 302a, such as proximal to the mount section 342 as shown. The insulating tube includes a pair of longitudinally spaced-apart circumferential recesses 345a, 345b. Each recess 345a, 345b has a corresponding windows 346a, 346b. Each of the windows 346a, 346b has a different circumferential orientation from the other of the windows, such that material from only one of the conductive halves 300a, 302a is exposed through the first window 346a, and such that material only from the other of the conductive halves 300a, 302a is exposed through the second window 346b. The exemplary embodiment shows the windows 346a, 346b having circumferential orientations offset by approximately 180 degrees.

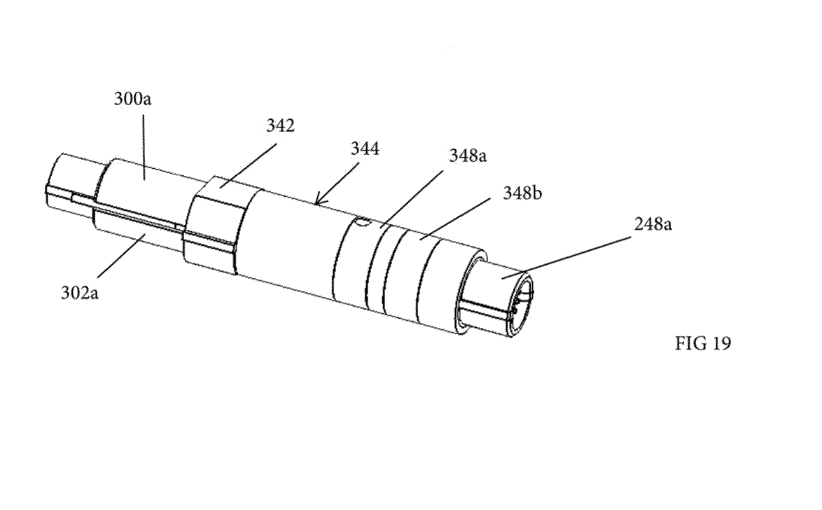

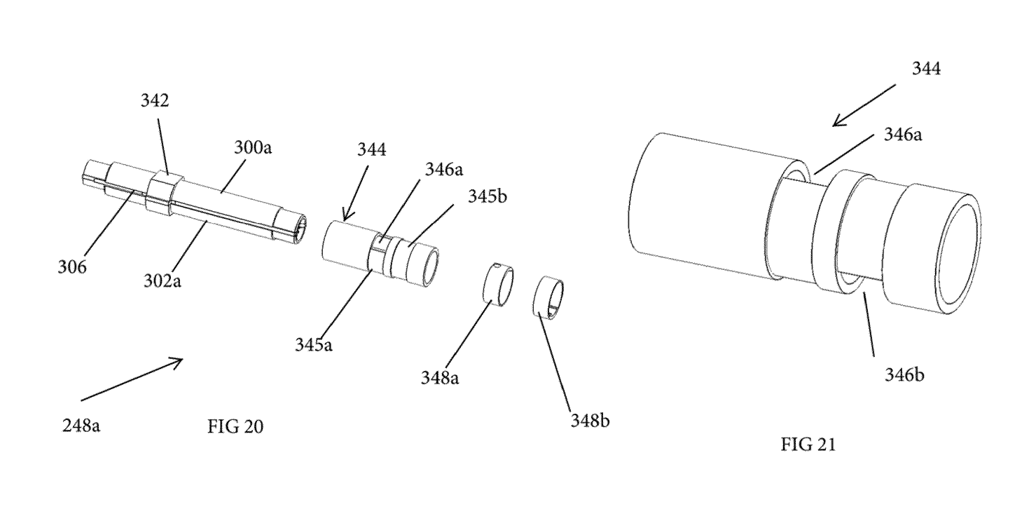

Electrically conductive ring contacts 348a, 348b are disposed in each of the recesses 345a, 345b, covering the corresponding ones of the windows. See FIG. 19. In the illustrated embodiment, window 346a is disposed over roll drive tube conductive half 300a, and ring 348a covers window 346a. As best shown in FIG. 16, conductive leaf spring 304a contacts ring 348a. Thus, energy from leaf spring 304a is conducted to roll drive tube conductive half 300a by ring 348a. This energy is further conducted to the instrument via whichever of the leads 310, 312 is associated with the fin of the instrument shaft that is positioned against the conductive half 300a. Similarly, window 346b is disposed over roll drive tube conductive half 302a, and ring 348b covers window 346. Conductive leaf spring 308a contacts ring 348b. Thus, energy from leaf spring 308a is conductive to roll drive tube conductive half 302a by ring 348b. This energy is further conducted to the instrument via whichever of the leads 310, 312 is associated with the fin of the instrument shaft that is positioned against the conductive half 302a.

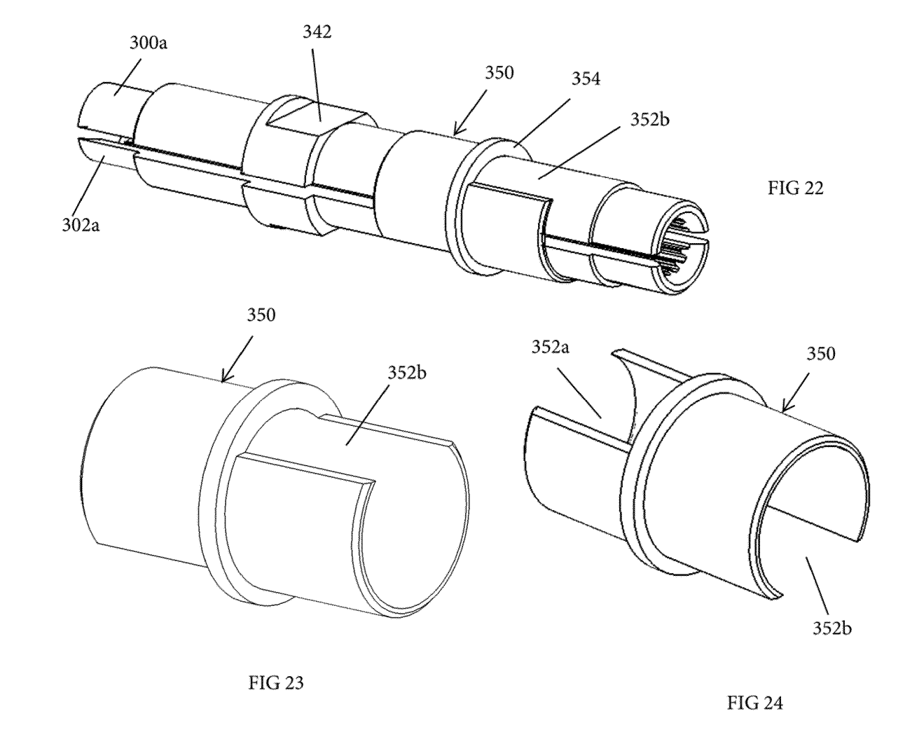

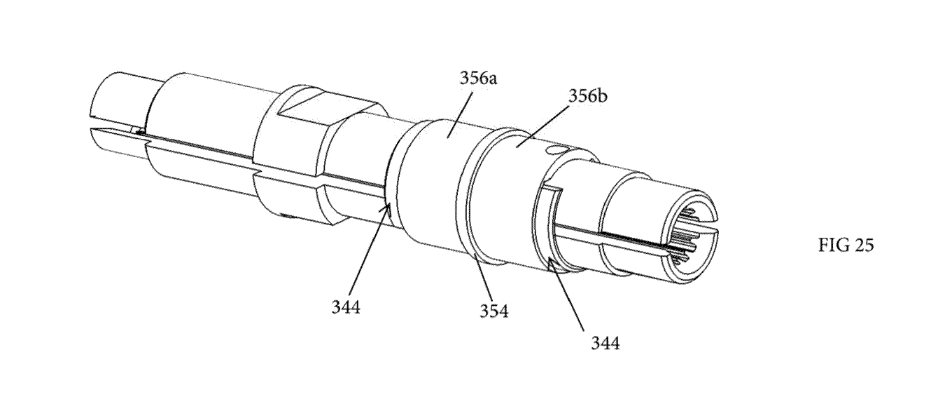

FIGS. 22 through 27 show an alternative arrangement of insulating and conductive elements arranged such that electrical energy from one of the leaf spring conductors passes only to one of conductive halves 300a, 302a of the roll drive tube, and such that electrical energy from the other one of the leaf spring conductors passes only to the other of the conductive halves.

Referring to FIG. 22, a first tubular insulator 350 is disposed over the roll drive tube. Tubular insulator 350 includes a proximal circumferential gap 352b and a distal circumferential gap 352a. Each of the gaps 352a, 352b has a different circumferential orientation from the other of the gaps, such that material from only one of the conductive halves 300a, 302a is exposed through the first gap 352a, and such that material only from the other of the conductive halves 300a, 302a is exposed through the second gap 352b. The exemplary embodiment shows the gaps 352a, 352b having circumferential orientations offset by approximately 180 degrees. Insulator 350 includes an annular rib 354 between the gaps 352a, 352b.

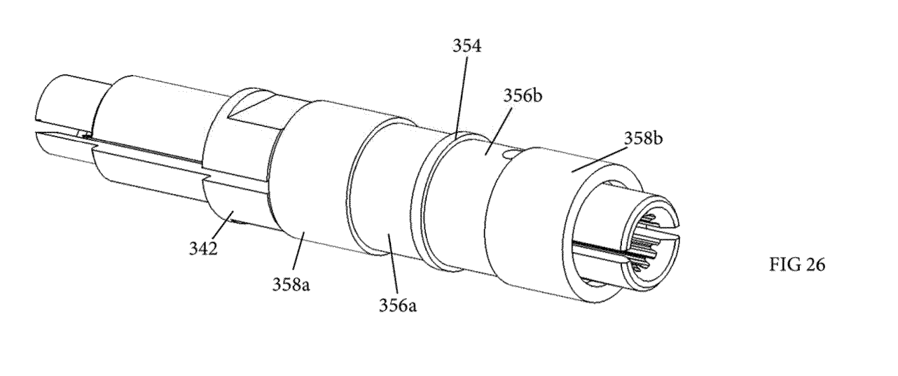

Electrically conductive ring contacts 356a, 356b are disposed over the tubular insulator 350 on opposite sides of the annular rib 354, such that each ring 356a, 356b covers a corresponding one of the gaps 352a, 352b. An insulative ring 358a is positioned distal to the ring 356a, and another is positioned proximal to the ring 356b as shown in FIG. 26.

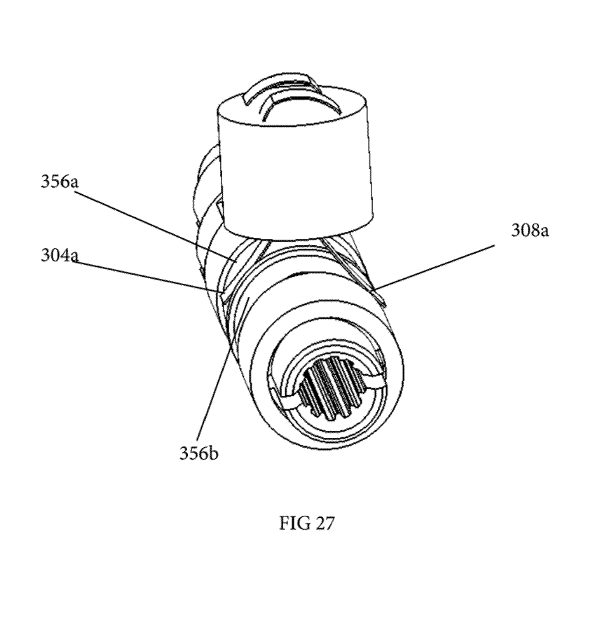

Thus, when fully assembled, gap 352a is disposed over roll drive tube conductive half 302a, and ring 356a covers gap 352a. Referring to FIG. 27, conductive leaf spring 304a contacts ring 356a. Thus, energy from leaf spring 304a is conducted to roll drive tube conductive half 302a by ring 356a. Similarly, gap 352b is disposed over roll drive tube conductive half 300a, and ring 356b covers gap 352b. Conductive leaf spring 308a contacts ring 356b. Thus, energy from leaf spring 308a is conductive to roll drive tube conductive half 302a by ring 356b.

Additional features may be included in the roll driver to enhance the use of the overall system. For example, an EEPROM having a usage counter may be position on the support base 334b such that it may be electronically coupled to the base unit 218 for incrementing the usage counter each time the roll driver is used.

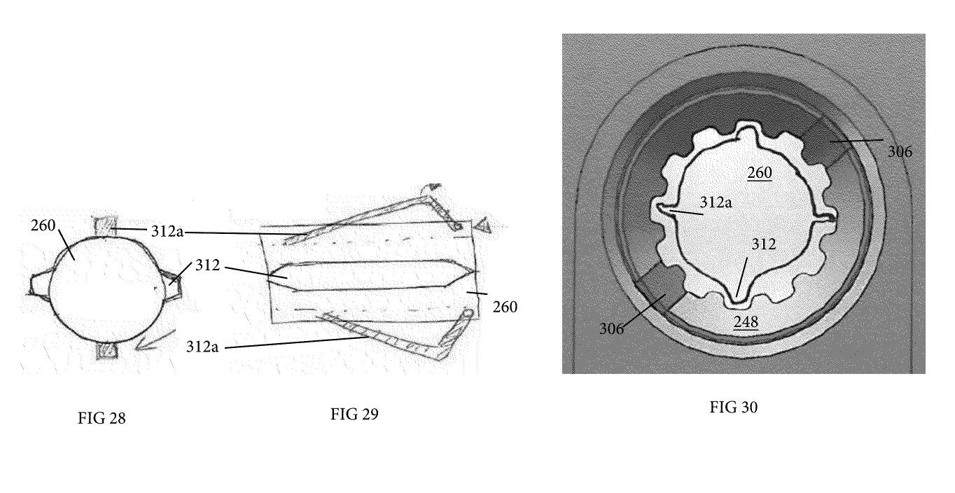

FIGS. 28-30 illustrate an alternate roll drive connect or drive segment 260. Referring to FIG. 28, this embodiment includes splines 312 that are similar to splines 256 shown in FIGS. 5A and 5B, which are rotationally engaged by elements of the roll drive tube 248 to cause rotation of the distal portion of the instrument. Two such splines are shown, spaced 180 degrees apart, although other numbers of splines could instead be used. This embodiment differs from the FIG. 5A-5B embodiment in that the drive segment 260 also includes electrically conductive leaf spring elements 312a. The leaf spring elements 312a are proportioned to make contact with the luminal wall of the roll drive tube 248 between the splines/ribs 258, preferably at its major diameter. As shown in FIG. 29, each leaf spring element 312a may be configured to have a first end (on the right in FIG. 29) attached to the drive segment 260, a second end that is unsecured, and a peak between the first and second ends. When the drive segment 260 is positioned in the roll drive tube, the peak of the leaf spring element 312a contacts the roll drive tube between adjacent splines/ribs 258, thus making electrical contact between the drive segment 260 and the roll drive tube. The bias of the spring helps to maintain this contact against the wall of the roll drive tube.

Referring to FIGS. 28 and 30, the splines 312 are wider than the leaf spring elements 312a, such that the splines 312 contact the splines/ribs 258 and bear the mechanical load when the drive segment 260 is axially rotated by the roll drive tube 248.

We claim:

1. A surgical system, comprising:
a surgical instrument having a shaft including a drive segment having a first conductive region, the shaft further including a first electrode and a first conductive path extending between the first conductive region and the first electrode;
a tubular socket having a conductive portion and a lumen for receiving the shaft, the lumen configured such that the drive segment rotationally engages with the tubular socket such that the first conductive region is in contact with the conductive portion of the tubular socket; and
a conductor in contact with an outer surface of the tubular socket, the conductor configured to receive electrical energy from an energy source, wherein the conductor, the first conductive region, and the conductive portion of the tubular socket are configured to conduct energy via the conductive path to the electrode.

2. The surgical instrument of claim 1, wherein tubular socket is axially rotatable and the conductor is positioned to maintain sliding contact with the outer surface during axial rotation of the tubular socket.

3. The system of claim 1, wherein:
the tubular socket includes a second conductive region, circumferentially spaced from the first conductive region, the first and second conductive regions circumferentially separated by insulative material;
the system further includes a second conductor in contact with an outer surface of the tubular socket, the second conductor configured to provide a return for the energy source, the first and second conductors positioned such that when the first conductor is in contact with the first conductive region the second conductor is in contact with the second conductive region;
the instrument includes a first electrode and a second electrode, the first conductive path associated with the first electrode;
the drive segment has a second conductive region and the instrument includes a second conductive path extending between the second conductive region and the second electrode.

4. The surgical instrument of claim 3, wherein tubular socket is axially rotatable and the first and second conductors are positioned to maintain sliding contact with the outer surface during axial rotation of the tubular socket.

5. A surgical instrument, comprising:
a shaft having a proximal shaft portion, a distal shaft portion having a distal electrode, and a drive segment disposed on the distal shaft portion such that axial rotation of the drive segment causes axial rotation of the distal shaft portion relative to the proximal shaft portion;
a first conductive region on the drive segment and an electrically conductive path extending between the first conductive region and the distal electrode.

6. The surgical instrument of claim 5, further including;
a second conductive region on the drive segment, the second conductive region circumferentially spaced and insulated from the first conductive region, and a second electrically conductive path extending between the first conductive region and a second electrode on the distal shaft portion.

7. The surgical instrument of claim 1, wherein the drive segment includes an outwardly biased conductive element positioned to maintain contact with a wall of the tubular socket.

8. The surgical instrument claim 7, wherein the outwardly biased conductive element is a conductive leaf spring.

9. The surgical instrument system of claim 1, wherein the system further includes a first connector in electrical communication with the conductor, the first connector configured to detachably connect to a cable extending from an energy source.

10. The surgical instrument system of claim 9, wherein the first connector includes a first position in electrical communication with the conductor and a second position electrically isolated from the conductor, the first connector selectively moveable between the first and second positions.

11. The surgical instrument system of claim 10, wherein the system further includes a second connector configured to detachably connect to a second cable extending from a second energy source, the second connector having first position in electrical communication with the conductor and a second position electrically isolated from the conductor, the first connector selectively moveable between the first and second positions.

12. A method of performing surgery, comprising:
providing a surgical instrument having a shaft including a drive segment having a first conductive region, the shaft further including a first electrode and a first conductive path extending between the first conductive region and the first electrode;
inserting the instrument into a lumen of a tubular socket and positioning the drive segment at least partially within the tubular socket such that the first conductive region is in contact with a conductive portion of the tubular socket;
selectively rotating the tubular socket, causing axial rolling of a distal portion of the surgical instrument;
electrically coupling an energy source to a conductor in contact with an outer surface of the tubular socket, such that the conductor, the first conductive region, and the conductive portion of the tubular socket conduct energy via the conductive path to the electrode.
